(19) United States
(12) Patent Application Publication
(10) Pub. No.: US 2003/0204237 A1
(43) Pub. Date: Oct. 30, 2003

(54) Stent delivery and aspiration catheter

(76) Inventors: Mike Krivoruchko, Forestville, CA (US); William Berthiaume, Santa Rosa, CA (US)

Correspondence Address: MEDTRONIC AVE, INC., 3576 UNOCAL PLACE, SANTA ROSA, CA, 95403, US

(21) Appl. No.: 10/133,139

(22) Filed: Apr. 25, 2002

(51) Int. Cl. A61F 2/06

(52) U.S. Cl. 623/1.11

(57) ABSTRACT

A stent delivery and aspiration system includes a pushrod having a tip flushing lumen in fluid communication with a porous tip, a stent over the pushrod, and a sheath which restrains the stent from expanding prior to deployment. The sheath including an aspiration tip in fluid communication with an aspiration lumen of the sheath, wherein after deployment of the stent, the sheath has an outer diameter less than an inner diameter of the stent. This allows the sheath to be moved forward into and through the stent enhancing the aspiration and removal of particulates within the vicinity of the deployed stent.

### BACKGROUND OF THE INVENTION

1. Field Of The Invention

The present invention relates to an intra-vascular device and method. More particularly, the present invention relates to a delivery system for deploying endoluminal prostheses within the lumens of the body and to a method of using the same.

2. Description Of The Related Art

Human blood vessels often become occluded or completely blocked by plaque, thrombi, other deposits, emboli or other substances, which reduce the blood carrying capacity of the vessel. Should the blockage occur at a critical place in the circulatory system, serious and permanent injury, or even death, can occur. To prevent this, some form of medical intervention is usually performed when significant occlusion is detected.

Various types of intervention techniques have been developed which facilitate the reduction or removal of the blockage in the blood vessel, allowing increased blood flow through the vessel. One technique for treating stenosis or occlusion of a blood vessel is the deployment of a stent. However, stent deployment inherently carries the risk of embolism caused by the dislodgement of the blocking material, which then moves downstream.

### SUMMARY OF THE INVENTION

In accordance with an embodiment of the present invention, a stent delivery and aspiration system includes a pushrod; a porous tip mounted to a distal end of the pushrod, the pushrod having a tip flushing lumen in fluid communication with the porous tip; a stent over the pushrod; and a sheath which restrains the stent from expanding prior to deployment. The sheath including an aspiration tip in fluid communication with an aspiration lumen of the sheath, wherein after deployment of the stent by retraction of the sheath, the sheath has an outer diameter less than an inner diameter of the stent.

Since the sheath has an outer diameter less than an inner diameter of the stent after deployment, the sheath can be moved forward into and through the stent. More particularly, the aspiration tip of the sheath is moved forward, and/or back and forth, within the stent thus enhancing the aspiration and removal of particulates within the vicinity of the deployed stent.

The present invention is best understood by reference to the following detailed description when read in conjunction with the accompanying drawings.

### BRIEF DESCRIPTION OF THE DRAWINGS

FIG. 1 is a side plan view, in partial cross-section, of a guide wire deployed within a parent artery or vessel of a patient adjacent to an occlusion in accordance with one embodiment of the present invention;

FIG. 2 is a side plan view, in partial cross-section, of the guide wire of FIG. 1 anchored within the vessel through inflation of an occlusion balloon of the guide wire in accordance with one embodiment of the present invention;

FIG. 3 is a side plan view, in partial cross-section, of a catheter over the guide wire of FIG. 2 and within the vessel in accordance with one embodiment of the present invention;

FIG. 4 is a side plan view, in partial cross-section, of the deployment of a stent of the catheter within the vessel in accordance with one embodiment of the present invention;

FIG. 5 is a side plan view, in partial cross-section, of the flushing of the vessel to remove particulates in accordance with one embodiment of the present invention;

FIG. 6 is a side plan view, in partial cross-section, of the flushing of the vessel to remove particulates in accordance with another embodiment of the present invention;

FIG. 7 is a side plan view, in partial cross-section, of the flushing of the vessel to remove particulates in accordance with yet another embodiment of the present invention; and

FIG. 8 is a side plan view, in partial cross-section, of the flushing of the vessel to remove particulates in accordance with yet another embodiment of the present invention.

Common reference numerals are used throughout the drawings and detailed description to indicate like elements.

### DETAILED DESCRIPTION

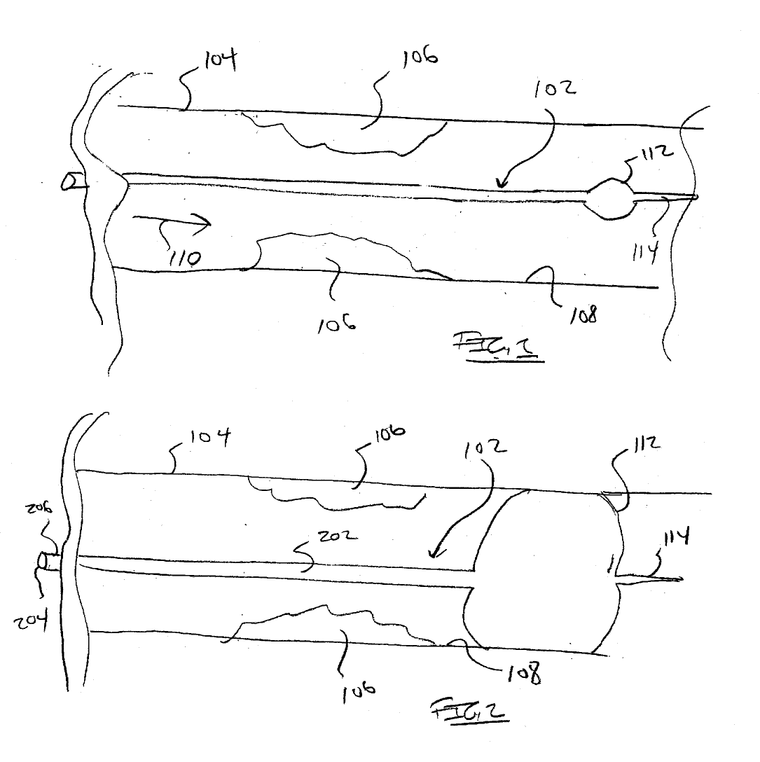

FIG. 1 is a side plan view, in partial cross-section, of a guide wire 102 deployed within a parent artery or vessel 104 of a patient adjacent to an occlusion 106 in accordance with one embodiment of the present invention. Occlusion 106 occludes or completely blocks blood flow through vessel 104. Illustratively, occlusion 106 is plaque, thrombi, other deposits, emboli or other substances on inner vessel wall 108 of vessel 104. Occlusion 106 reduces the blood carrying capacity of vessel 104. Left untreated, occlusion 106 could cause serious and permanent injury, or even death to the patient.

Blood flow through vessel 104 is in the direction indicated by arrow 110. Guide wire 102 is introduced intra-vascularly and guided through vessel 104 to occlusion 106 using any one of a number of techniques well known to those of skill in the art.

As shown in FIG. 1, guide wire 102 includes an anchoring device 112 at a distal end 114 of guide wire 102. Guide wire 102 is guided through vessel 104 such that anchoring device 112 is located downstream from occlusion 106, i.e., in the direction of blood flow from occlusion 106.

FIG. 2 is a side plan view, in partial cross-section, of guide wire 102 of FIG. 1 anchored within vessel 104 through inflation of anchoring device 112 of guide wire 102 in accordance with an embodiment of the present invention. Referring now to FIGS. 1 and 2 together, in accordance with this embodiment, anchoring device 112 is an occlusion balloon, sometimes called an occlusion balloon 112. Occlusion balloon 112 is inflated, sometimes called expanded.

More particularly, guide wire 102 includes an occlusion balloon lumen 202 in fluid communication with occlusion balloon 112. Occlusion balloon 112 is filled with a fluid provided through occlusion balloon lumen 202. However, in another embodiment, occlusion balloon lumen 202 and occlusion balloon 112 are separate structures over guide wire 102, instead of being parts of guide wire 102.

This fluid is provided to occlusion balloon lumen 202 through an occlusion balloon lumen port 204 of occlusion balloon lumen 202 located at a proximal end 206 of occlusion balloon lumen 202 using any one of a number of techniques well known to those of skill in the art. The particular technique used is not essential to the invention.

Once inflated as illustrated in FIG. 2, occlusion balloon 112 is anchored against inner vessel wall 108. Consequently, guide wire 102 is fixedly positioned within vessel 104. Further, inflation of occlusion balloon 112 seals vessel 104 and thus blocks blood flow through vessel 104.

However, in another embodiment, anchoring device 112 is permeable to blood, e.g., is a filter, such that blood flow continues through vessel 104 even after anchoring device 112 is anchored against inner vessel wall 108. However, anchoring device 112 filters and captures any particulates contained within the blood flowing through anchoring device 112 in accordance with this embodiment. Further, in one embodiment when anchoring device 112 is a filter, instead of guide wire 102 having occlusion balloon lumen 202 as discussed above, guide wire 102 includes a mechanical device such as a wire (or filter tube/sheath) which facilitates deployment of a collapsible anchoring device 112.

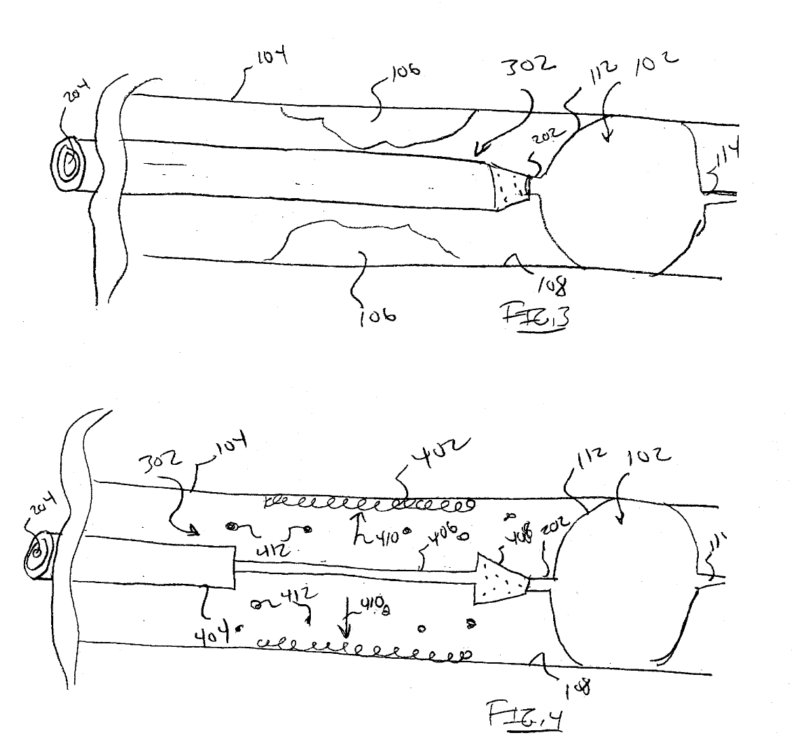

FIG. 3 is a side plan view, in partial cross-section, of a stent delivery and aspiration catheter 302 over guide wire 102 of FIG. 2 and within vessel 104 in accordance with one embodiment of the present invention. Referring now to FIGS. 2 and 3 together, catheter 302 is introduced intra-vascularly and guided to occlusion 106 by guide wire 102. In one embodiment, catheter 302 includes radiopaque markers (not shown) which facilitate positioning and tracking of catheter 302. Catheter 302 and guide wire 102 collectively are sometimes referred to as a stent delivery and aspiration system.

FIG. 4 is a side plan view, in partial cross-section, of the deployment of a stent 402 of catheter 302 within vessel 104 in accordance with one embodiment of the present invention. Referring now to FIGS. 3 and 4 together, catheter 302 includes a sheath 404, a pushrod 406, and a porous tip 408.

Prior to deployment, stent 402 is placed over pushrod 406 and is radially compressed and restrain from expanding within sheath 404. Catheter 302 is positioned such that stent 402 is delivered to and located within occlusion 106. Sheath 404 is retracted deploying stent 402 as indicated by the arrows 410.

In one embodiment, stent 402 is self-expandable and thus self expands upon retraction of sheath 404. However, in another embodiment, stent 402 is placed over a catheter balloon, for example, on or integral with pushrod 406. This catheter balloon is expanded to expand stent 402. After deployment of stent 402, this catheter balloon is deflated.

After deployment of stent 402, stent 402 is anchored to inner vessel wall 108. Stent 402 compresses occlusion 106 thus providing a larger pathway through vessel 104. Stated another way, stent 402 opens vessel 104.

However, as a result of the above procedure, particulates 412, e.g., debris and pieces from occlusion 106, are introduced into vessel 104. To prevent particulates 412 from escaping into the vasculature of the patient and the associated complications, in accordance with various embodiments of the present invention, particulates 412 are captured and removed from the patient as discussed further below.

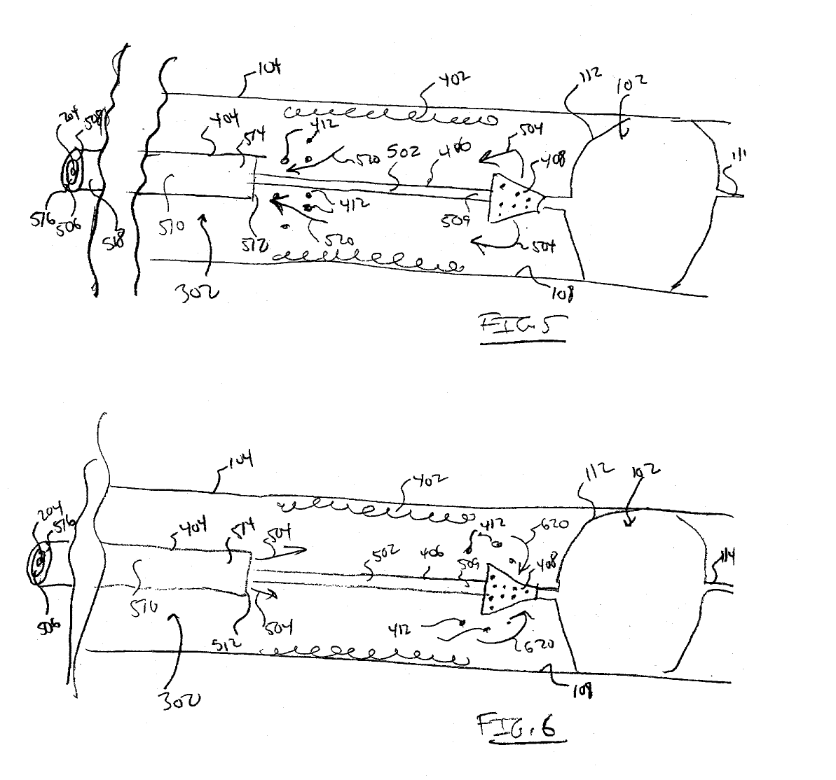

FIG. 5 is a side plan view, in partial cross-section, of the flushing of vessel 104 to remove particulates 412 in accordance with one embodiment of the present invention. Referring now to FIG. 5, pushrod 406 includes a tip flushing lumen 502. Porous tip 408 is mounted to a distal end 509 of pushrod 406. Further, porous tip 408 is porous and in fluid communication with tip flushing lumen 502.

Illustratively, porous tip 408 includes pores, channels or other passages through which a flushing fluid can pass. In this manner, a flushing fluid 504 provided through tip flushing lumen 502 of pushrod 406 is passed through porous tip 408 and into vessel 104.

Flushing fluid 504 is provided to tip flushing lumen 502 through a tip flushing lumen port 506 of tip flushing lumen 502 located at a proximal end 508 of tip flushing lumen 502 using any one of a number of techniques well known to those of skill in the art. The particular technique used is not essential to the invention.

Further, sheath 404 includes an aspiration lumen 510 and an aspiration tip 512 at a distal end 514 of sheath 404. In one embodiment, aspiration tip 512 is simply the end, sometimes called opening, of sheath 404. Aspiration tip 512 is in fluid communication with aspiration lumen 510.

During use, suction is provided through aspiration lumen 510 of sheath 404 and to aspiration tip 512. This suction is provided to aspiration lumen 510 through an aspiration lumen port 516 of aspiration lumen 510 located at a proximal end 518 of aspiration lumen 510 using any one of a number of techniques well known to those of skill in the art. The particular technique used is not critical as long as the desired result is achieved. Aspiration tip 512 is in fluid communication with aspiration lumen port 516. In the above manner, fluid in vessel 104 is aspirated through aspiration tip 512 and into aspiration lumen 510 as indicated by the arrows 520.

As a result of flushing fluid 504 provided through porous tip 408 and the aspiration provided through aspiration tip 512, vessel 104 is flushed within the vicinity of stent 402. More particularly, a flushing flow between porous tip 408 and aspiration tip 512 flushes particulates 412 from vessel 104 and into aspiration tip 512. However, in alternative embodiments, either flushing fluid 504 is provided through porous tip 408 or the aspiration is provided through aspiration tip 512 but not both.

In accordance with one embodiment, particulates 412 located at or near anchoring device 112 are removed in the above manner. For example, when anchoring device 112 is a filter, which has been clogged with particulates 412 such that blood flow through vessel 104 is diminished or stopped, particulates 412 are removed to restore blood flow through vessel 104.

FIG. 6 is a side plan view, in partial cross-section, of the flushing of vessel 104 to remove particulates 412 in accordance with another embodiment of the present invention. Referring now to FIG. 6, in accordance with this embodiment, flushing fluid 504 is provided through aspiration lumen 510 of sheath 404 and to aspiration tip 512, sometimes called tip 512. Flushing fluid 504 is passed through aspiration tip 512 and into vessel 104. Flushing fluid 504 is provided to aspiration lumen 510 through aspiration lumen port 516.

During use, suction is provided through tip flushing lumen 502 of pushrod 406 and to porous tip 408. This suction is provided to tip flushing lumen 502 through tip flushing lumen port 506 of tip flushing lumen 502. In the above manner, fluid in vessel 104 is aspirated through porous tip 408 and into tip flushing lumen 502 as indicated by the arrows 620.

As a result of flushing fluid 504 provided through tip 512 and the aspiration provided through porous tip 408, vessel 104 is flushed within the vicinity of stent 402. More particularly, a flushing flow between tip 512 and porous tip 408 flushes particulates 412 from vessel 104 and into porous tip 408. However, in alternative embodiments, either flushing fluid 504 is provided through tip 512 or the aspiration is provided through porous tip 408 but not both.

In accordance with one embodiment, particulates 412 located at or near anchoring device 112 are removed in the above manner. For example, when anchoring device 112 is a filter, which has been clogged with particulates 412 such that blood flow through vessel 104 is diminished or stopped, particulates 412 are removed to restore blood flow through vessel 104.

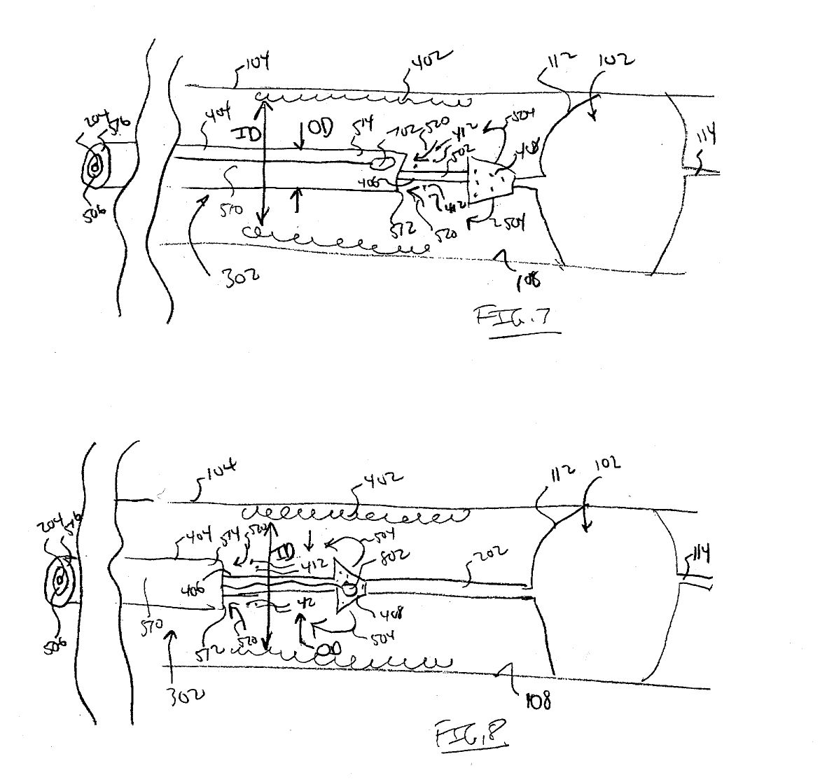

FIG. 7 is a side plan view, in partial cross-section, of the flushing of vessel 104 to remove particulates 412 in accordance with yet another embodiment of the present invention. FIG. 7 is similar to FIG. 5 and only the significant differences are discussed below.

Referring now to FIG. 7, in accordance with this embodiment, sheath 404 and aspiration tip 512 have an outer diameter OD less than an inner diameter ID of stent 402 after deployment, i.e., after stent 402 expanded. This allows sheath 404 to be moved forward into and through stent 402. More particularly, aspiration tip 512 is moved forward, and/or back and forth, within stent 402 thus enhancing the aspiration and removal of particulates 412.

In accordance with one embodiment, aspiration tip 512 is moved forward into and through stent 402 to be adjacent anchoring device 112. Accordingly, particulates 412 located at or near anchoring device 112 are effectively and reliably removed. For example, when anchoring device 112 is a filter, which has been clogged with particulates 412 such that blood flow through vessel 104 is diminished or stopped, particulates 412 are removed to restore blood flow through vessel 104 in the above manner.

Also in accordance with this embodiment, a vibrator 702, e.g., an ultrasonic generator or piezoelectric oscillator, is mounted to distal end 514 of sheath 404 adjacent aspiration tip 512. Vibrator 702 is activated to vibrate distal end 514 of sheath 404 and aspiration tip 512. This scours vessel 104 and enhances the dislocation and capture of particulates 412.

FIG. 8 is a side plan view, in partial cross-section, of the flushing of vessel 104 to remove particulates 412 in accordance with yet another embodiment of the present invention. FIG. 8 is similar to FIG. 5 and only the significant differences are discussed below.

Referring now to FIG. 8, in accordance with this embodiment, porous tip 408 has a maximum outer diameter OD less than inner diameter ID of stent 402 after deployment, i.e., after stent 402 expanded. This allows porous tip 408 to be moved into and through stent 402. More particularly, porous tip 408 is moved backward, and/or back and forth, within stent 402 thus enhancing the aspiration and removal of particulates 412.

Also in accordance with this embodiment, a vibrator 802 is mounted to porous tip 408. Vibrator 802 is activated to vibrate porous tip 408. This scours vessel 104 and enhances the dislocation and capture of particulates 412.

In FIGS. 7 and 8, flushing fluid 504 is provided through porous tip 408 and aspiration is provided through aspiration tip 512 to remove particulates 412. However, is other embodiments, flushing fluid 504 is provided through aspiration tip 512 and aspiration is provided through porous tip 408 in the arrangements of FIGS. 7 and 8 in a manner similar to that discussed above in reference to FIG. 6. In other embodiments, either flushing fluid 504 or aspiration is provided but not both.

After removal of particulates 412 as discussed above, catheter 302 is removed from the patient. Anchoring device 112 is deflated and guide wire 102 is also removed from the patient.

This disclosure provides exemplary embodiments of the present invention. The scope of the present invention is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A stent delivery and aspiration system comprising:
a catheter comprising a sheath and a stent, wherein said sheath restrains said stent during delivery of said stent;
wherein said sheath comprising an aspiration tip in fluid communication with an aspiration lumen port; and
wherein after deployment of said stent, said sheath is sized to pass through said stent.

2. The stent delivery and aspiration system of claim 1 wherein said stent is self-expanding.

3. The stent delivery and aspiration system of claim 1 wherein said stent self-expands upon retraction of said sheath.

4. The stent delivery and aspiration system of claim 1 wherein said sheath comprises an outer diameter less than an inner diameter of said stent after deployment.

5. The stent delivery and aspiration system of claim 1 further comprising a guide wire, said catheter being located over said guide wire.

6. The stent delivery and aspiration system of claim 5 wherein said guide wire comprises an anchoring device for anchoring said guide wire within a vessel.

7. The stent delivery and aspiration system of claim 6 wherein said anchoring device comprises an occlusion balloon.

8. The stent delivery and aspiration system of claim 7 wherein said guide wire comprises an occlusion balloon lumen.

9. The stent delivery and aspiration system of claim 6 wherein said anchoring device comprises a filter.

10. The stent delivery and aspiration system of claim 1 further comprising a vibrator mounted to said sheath adjacent said aspiration tip.

11. The stent delivery and aspiration system of claim 1 wherein said catheter further comprises a porous tip in fluid communication with a tip flushing lumen port.

12. The stent delivery and aspiration system of claim 11 wherein said porous tip is porous to allow a flushing fluid to pass therethrough.

13. The stent delivery and aspiration system of claim 11 further comprising a vibrator mounted to said porous tip.

14. The stent delivery and aspiration system of claim 11 wherein said porous tip comprises an outer diameter less than an inner diameter of said stent after deployment.

15. A stent delivery and aspiration system comprising:
a pushrod;
a porous tip mounted to a distal end of said pushrod, said pushrod comprising a tip flushing lumen in fluid communication with said porous tip;
a stent over said pushrod; and
a sheath which restrains said stent from expanding prior to deployment, said sheath comprising an aspiration tip in fluid communication with an aspiration lumen of said sheath, wherein after deployment of said stent by retraction of said sheath, said sheath has an outer diameter less than an inner diameter of said stent.

16. The stent delivery and aspiration system of claim 15 further comprising a vibrator mounted to said sheath adjacent said aspiration tip.

17. The stent delivery and aspiration system of claim 15 further comprising a vibrator mounted to said porous tip.

18. A method comprising:
restraining a stent within a sheath;
delivering said stent to an occlusion within a vessel;
deploying said stent by retracting said sheath, wherein said stent expands upon said deploying;
moving said sheath forward and into said stent; and
providing suction to an aspiration tip of said sheath.

19. The method of claim 18 wherein said providing suction comprises aspirating fluid in said vessel.

20. The method of claim 19 wherein said providing suction further comprises aspirating particulates in said vessel.

21. The method of claim 18 further comprising scouring said vessel by vibrating said sheath.

22. The method of claim 18 further comprising moving said aspiration tip back and forth within said stent.

23. The method of claim 18 further comprising providing a flushing fluid to a porous tip to flush said vessel within a vicinity of said stent.

24. The method of claim 18 further comprising anchoring a guide wire within said vessel, wherein said delivering said stent comprises guiding a catheter comprising said stent over said guide wire.

25. A method comprising:
restraining a stent over a pushrod;
delivering said stent to an occlusion within a vessel;
deploying said stent, wherein said stent expands upon said deploying;
moving a porous tip mounted to said pushrod into said stent; and
providing suction to said porous tip.

26. A method comprising:
restraining a stent within a sheath;
delivering said stent to an occlusion within a vessel;
deploying said stent by retracting said sheath, wherein said stent expands upon said deploying;
moving said sheath forward and into said stent; and
providing a flushing fluid to a tip of said sheath.

27. A method comprising:
restraining a stent over a pushrod;
delivering said stent to an occlusion within a vessel;
deploying said stent, wherein said stent expands upon said deploying;
moving a porous tip mounted to said pushrod into said stent; and
providing a flushing fluid to said porous tip.
